(19) United States
(12) Patent Application Publication
(10) Pub. No.: US 2025/0010012 A1
(43) Pub. Date: Jan. 9, 2025

(54) RESPIRATORY APPARATUS

(71) Applicant: BANYAN LICENSING L.L.C., Vero Beach, FL (US)

(72) Inventors: Sanya Karwani, Charlotte, NC (US); E. Scott Davis, Vero Beach, FL (US)

(73) Assignee: BANYAN LICENSING L.L.C., Vero Beach, FL (US)

(21) Appl. No.: 18/716,076

(22) PCT Filed: Dec. 1, 2022
(86) PCT No.: PCT/US2022/051536

§ 371 Date: Jun. 3, 2024

Related U.S. Application Data
(60) Provisional application No. 63/284,969, filed on Dec. 1, 2021.

(51) Int. Cl. A61M 16/08; A61G 13/12; A61M 25/02

(52) U.S. Cl. CPC A61M 16/0875; A61G 13/1215; A61M 2025/024

(57) ABSTRACT

An apparatus is provided for routing hose, comprising a support device, wherein the support device defines a first surface and an opposite second surface, a first end and an opposite second end. The support device comprising an aperture proximate the first surface extending from the first surface to the second surface; and a channel aperture extending from the first end toward the second end, wherein the channel aperture at least partially defines the second surface; and a resilient member.

### FIELD OF THE INVENTION

The present invention relates to respiratory apparatuses and, more specifically, an apparatus that is structured to route respiratory gas through a support device while providing the user with a flow of gas through a hose device.

### BACKGROUND OF THE INVENTION

A support device is generally useful for the purpose of rendering comfort or support to a user. Respiratory devices, including but not limited to continuous positive airway pressure devices (“CPAP”) are useful for providing a user with pressurized air, such that the user is able to sleep without the user's airways collapsing. Conventional respiratory devices utilize pressurized hoses which are routed from the nasal area of a user downwards to an air pump. Since these hoses may interfere with the user's bedding, pillows, arms, legs, and the like, the hoses cause discomfort and irritation to the user while the user sleeps. Thus, there is a need for a respiratory apparatus that is structured to comfortably route the hoses away from the user.

### SUMMARY OF THE INVENTION

An apparatus is provided for routing hose in a respiratory device. According to one embodiment, the apparatus comprises a support device and a resilient member, wherein the support device defines a first surface and an opposite second surface, a first end and an opposite second end. In one embodiment, the support device comprises an aperture proximate the first surface extending from the first surface to the second surface; and a channel aperture extending from the first end toward the second end, wherein the channel aperture at least partially defines the second surface. In one embodiment, the apparatus comprises a hose having an outer surface and the resilient member defines an inner surface, and wherein the inner surface of the resilient member is coupled to the outer surface of the hose. In another embodiment, the apparatus the hose has a first section and a second section, wherein the first and second sections have different flexibility. In another embodiment, the first section comprises a flexible hose and the second section comprises a vertical hose. In one embodiment, the vertical hose is positioned within the aperture. In another embodiment, the flexible hose is positioned within the aperture. In another embodiment, the apparatus comprises an extension hose and an angle fitting, and wherein the flexible hose is coupled to the vertical hose, and the angle fitting is coupled to the vertical hose and the extension hose. In another embodiment, the apparatus comprises an extension hose, wherein the extension hose is positioned proximate the channel aperture. In another embodiment, the resilient member is positioned within the aperture and is positioned proximate the channel aperture. In another embodiment, the resilient member is releasably coupled to the flexible hose. In another embodiment, the resilient member comprises a plurality of a ball-and-socket joints,

### BRIEF DESCRIPTION OF THE DRAWINGS

The respiratory apparatus of the present invention alleviates the forgoing deficiencies of conventional support devices and also provides additional advantages. The advantages and features of the invention, and the manner in which the same are accomplished, will become more readily apparent upon consideration of the following detail description of the invention taken in conjunction with the accompanying drawings, which illustrate preferred and exemplary embodiments and which are not necessarily drawn to scale, wherein:

FIG. 1a illustrates a first perspective view 10a of the respiratory apparatus 100, in accordance with one embodiment of the invention;

FIG. 1b illustrates a second perspective view 10b of the respiratory apparatus 100, in accordance with the embodiment of FIG. 1a;

FIG. 1c illustrates a rear view 10c of the respiratory apparatus 100, in accordance with the embodiment of FIG. 1a;

FIG. 1d illustrates a right-side view 10d of the respiratory apparatus 100, in accordance with the embodiment of FIG. 1a;

FIG. 1e illustrates a cross-section view 10e of the respiratory apparatus 100, in accordance with the embodiment of FIG. 1a;

FIG. 1f illustrates a top plan view 10f of the respiratory apparatus 100, in accordance with the embodiment of FIG. 1a;

FIG. 2a illustrates a first perspective view 20a of the respiratory apparatus 200, in accordance with one embodiment of the invention;

FIG. 2b illustrates a second perspective view 20b of the respiratory apparatus 200, in accordance with the embodiment of FIG. 2a;

FIG. 3a illustrates a first perspective view 30a of the respiratory apparatus 300, in accordance with another embodiment of the invention;

FIG. 3b illustrates a second perspective view 30b of the respiratory apparatus 300, in accordance with the embodiment of FIG. 3a;

FIG. 3c illustrates a rear view 30c of the respiratory apparatus 300, in accordance with the embodiment of FIG. 3a;

FIG. 3d illustrates a right-side view 30d of the respiratory apparatus 300, in accordance with the embodiment of FIG. 3a;

FIG. 3e illustrates a cross-section view 30e of the respiratory apparatus 300, in accordance with the embodiment of FIG. 3a;

FIG. 3f illustrates a top plan view 30f of the respiratory apparatus 300, in accordance with the embodiment of FIG. 3a;

FIG. 4 illustrates a perspective view 40 of the respiratory apparatus 400, in accordance with another embodiment of the invention;

FIG. 5 illustrates a perspective view 50 of the respiratory apparatus 500, in accordance with another embodiment of the invention;

FIG. 6a illustrates a first cross section view 60a of the respiratory apparatus 600, in accordance with another embodiment of the invention;

FIG. 6b illustrates a top plan view 60b of the respiratory apparatus 600, in accordance with the embodiment of FIG. 6a;

FIG. 6c illustrates a rear view 60c of the respiratory apparatus 600, in accordance with the embodiment of FIG. 6a;

FIG. 6d illustrates a right-side view 60d of the respiratory apparatus 600, in accordance with the embodiment of FIG. 6a;

FIG. 6e illustrates a second cross-section view 60e of the respiratory apparatus 600, in accordance with the embodiment of FIG. 6a;

FIG. 6f illustrates a first perspective view 60f of the respiratory apparatus 600, in accordance with the embodiment of FIG. 6a;

FIG. 6g illustrates a second perspective view 60f of the respiratory apparatus 600, in accordance with the embodiment of FIG. 6a;

FIG. 7a illustrates a rear view 70a of the respiratory apparatus 700, in accordance with another embodiment of the invention;

FIG. 7b illustrates a right-side view 70b of the respiratory apparatus 700, in accordance with the embodiment of FIG. 7a;

FIG. 7c illustrates a cross section 70c of the respiratory apparatus 700, in accordance with the embodiment of FIG. 7a;

FIG. 7d illustrates a first perspective view 70d of the respiratory apparatus 700, in accordance with the embodiment of FIG. 7a;

FIG. 7e illustrates a second perspective view 70e of the respiratory apparatus 700, in accordance with the embodiment of FIG. 7a;

FIG. 8 illustrates a perspective view 80 of the respiratory apparatus 800, in accordance with another embodiment of the invention;

FIG. 9 illustrates a perspective view 90 of the respiratory apparatus 900, in accordance with another embodiment of the invention;

FIG. 10a illustrates a right-side cross-section view 100a of the respiratory apparatus 1000a, in accordance with another embodiment of the invention;

FIG. 10b illustrates a right-side cross-section view 100b of the respiratory apparatus 1000b, in accordance with another embodiment of the invention;

FIG. 10c illustrates a right-side cross-section view 100c of the respiratory apparatus 1000c, in accordance with another embodiment of the invention;

FIG. 10d illustrates a right-side cross-section view 100d of the respiratory apparatus 1000d, in accordance with another embodiment of the invention;

FIG. 11a illustrates a perspective view 110a of the resilient member 1106, in accordance with another embodiment of the invention;

FIG. 11b illustrates a perspective view 110b of the resilient member 1108, in accordance with another embodiment of the invention;

FIG. 11c illustrates a perspective view 110b of the resilient member 1110, in accordance with another embodiment of the invention;

FIG. 12a illustrates a right-side cross-section view 120a of the respiratory apparatus 1200, in accordance with another embodiment of the invention;

FIG. 12b illustrates a perspective view 120b of the respiratory apparatus 1200, in accordance with the embodiment of FIG. 12a;

FIG. 13a illustrates a rear view 130a of the respiratory apparatus 1300, in accordance with another embodiment of the invention;

FIG. 13b illustrates a right-side view 130b of the respiratory apparatus 1300, in accordance with the embodiment of FIG. 13a;

FIG. 13c illustrates a cross-section view 130c of the respiratory apparatus 1300, in accordance with the embodiment of FIG. 13a; and

FIG. 13d illustrates a top plan view 130d of the respiratory apparatus 1300, in accordance with the embodiment of FIG. 13a.

### DETAILED DESCRIPTION OF THE INVENTION

The present invention now will be described more fully hereinafter with reference to the accompanying drawings, in which some, but not all embodiments of the invention are shown. This invention may be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

A respiratory device is a medical device designed to provide the flow gas either to or from the oral and nasal cavities of a user during a user's sleep or rest. The gas may comprise oxygen, air, carbon dioxide, nitrogen, or the like. Generally, the present invention may be structured to accommodate any number of respiratory devices. Specifically, it may be structured for a CPAP device, however other embodiments and usages are considered. For example, the invention may be implemented in hospitals and ambulances with respect to providing supplemental oxygen to a patient, intubating a patient, feeding tubes, respirators, or the like. Conventional respiratory devices do not integrate with sleeping apparatuses such as pillows, bedding, or the like. As such, when a respiratory device is applied to the face region of a user while the user lies in a bed, chair, or stretcher (or any other situation in which a support device may be used), the hose portions of the respiratory device may become entangled in the bedding, extremities of the user such as arms and legs, clothing, pets, bed partners, or the like, and could lead to discomfort and/or irritation.

As used herein, a “respiratory device” may refer to a CPAP machine, ventilator, oxygen tank, positive airway pressure machine (“PAP”), bi-level positive airway pressure machine (“BiPAP”), anesthesia delivery system, or the like.

As used herein, a “support device” may refer to a device structured to support and/or route various respiratory device accompaniments such as tubes, hoses, conduits, and so forth. In some embodiments, the support device may comprise a pillow, mattress, mattress topper, standalone device used alongside a pillow or a mattress, or any number of surface elements or devices configured to accept the components described herein. As such, the support device may be used to support the body of the user or one or more portions of the body of the user. However, in other embodiments the support device may be primarily structured for supporting and/or routing various respiratory device accompaniments, and not be structured to primarily support portions of the body of the user. For example, the support device may be placed behind a pillow or mattress, on the headboard of a bed, wall, ground, bed-side table, and so forth.

The respiratory apparatus of the embodiments of the present invention, as will be described in detail below, overcomes the foregoing disadvantages of the traditional respiratory device hose routing methods. The present invention integrates the respiratory device's hose into a support device, such that the hose is presented to the user vertically above the user's face. By positioning the hose in this area, the length of exposed hose is minimized in order to reduce the capacity for the hose to become entangled in any intermediary object. Furthermore, since the hose is generally routed upwards above the user's face, behind the user's head, and then downwards through the support device itself, the hose has now formed an integral unit with the support device, and as such, the user may adjust the positioning of the support device before or during sleep such that the hose (and thereby the user's face) experiences far less strain than occurs when moving the hose of the conventional system. Some embodiments of the invention comprise an adjustable resilient member, which allows the user to articulate the positioning of the hose to a position in which it is most comfortable to the user, and the adjustable resilient member will maintain this position. The apparatus routes the hose through the surface of the support device proximate the user, and subsequently routes the hose along a 90 degree bend outwards toward the end of the support device, with the hose being recessed within a channel-like void of the underside of the support device. The apparatus is configurable to allow the hose to exit either side of the support device, such that the user may adapt the apparatus to the user's sleeping environment. Moreover, some embodiments of the respiratory device have specific contours and predetermined thickness profiles, allowing the portion of the flexible hose closest to the face of the user to rest within one or more contours to allow the user to adopt a side-sleeping position easily and comfortably. The functions and features of various embodiments of the respiratory apparatus are described below. It should be appreciated that these features can be provided separately in respiratory apparatus or the respiratory apparatus may have combinations of individual features or may have all of the features.

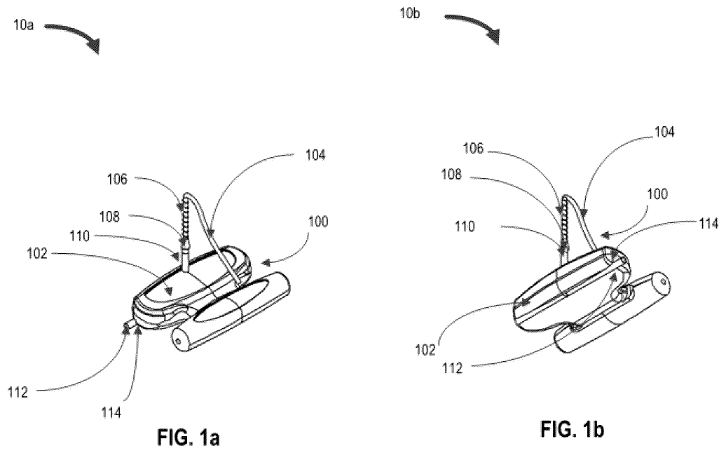

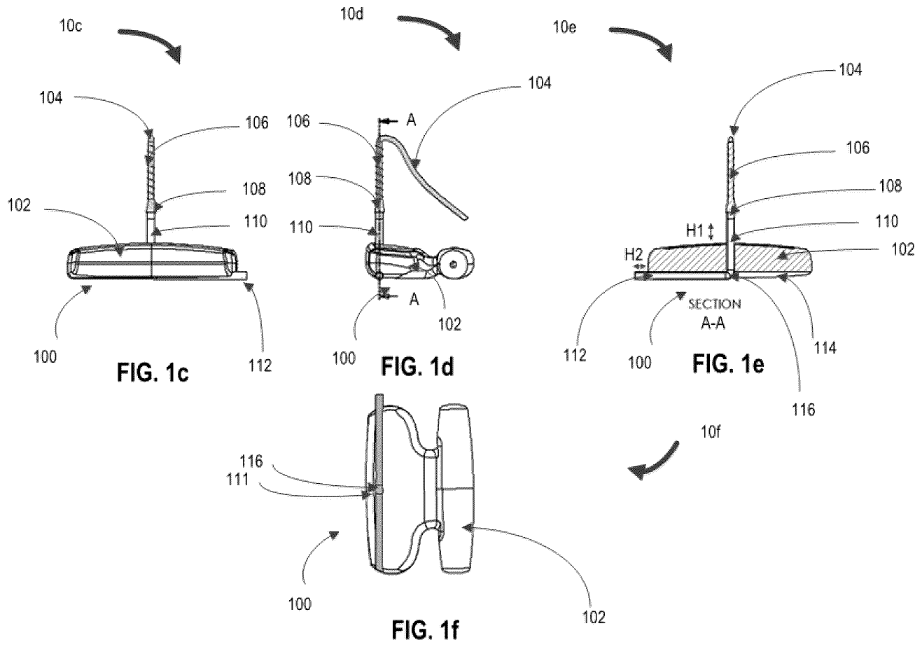

FIGS. 1a-1f, FIGS. 2a-2b, and FIGS. 6a-6g illustrate non-limiting examples of a respiratory apparatuses 100, 200, and 600, respectively, in accordance with some embodiments of the invention. The respiratory apparatuses 100, 200, 600 comprises a support device 102, as illustrated by FIGS. 1a-1f.

In some embodiments, the respiratory apparatuses 100, 200, 600 also comprises an outer cover (not illustrated) configured to at least partially enclose the support device 102. The outer cover is preferably made of a material that is soft to the skin of the user, e.g., silk, satin or cotton, and is preferably removable so that the outer cover may be washed and then refitted on the support device. In some embodiments, the support device 102 comprises one or more types of filler material, which may be enclosed by a barrier or sheath (not illustrated) in some instances. The configuration of these components is described below in detail. The respiratory apparatuses 100, 200, 600 can comprise more or fewer components as required for various embodiments.

Referring to FIGS. 1a-1f, FIGS. 2a-2b, and FIGS. 6a-6g, the support device 102 of the respiratory apparatuses 100, 200, 600, respectively, is shaped, dimensioned and otherwise structured to support and/or route various respiratory device accompaniments such as tubes, hoses, conduits, and so forth. In some embodiments of the invention, support device 102 may be structured to provide cushioning to, or cushioned protection from, the head and/or neck of a user. For example, in some embodiments, user places the respiratory apparatuses 100, 200, 600 under the user's head and/or neck, during a back-sleeping or side-sleeping position. In other embodiments, respiratory apparatuses 100, 200, 600 may be placed in a location remote to the user, such that the user does not interact with support device 102 during normal usage. For example, respiratory apparatuses 100, 200, 600 may be placed behind a pillow or mattress, on the headboard of a bed, wall, ground, or bed-side table.

In some embodiments of the present invention, the support device 102 may be substantially rectangular cuboid in shape, whereas in other embodiments, support device 102 may comprise complex curvatures structures to provide additional support to the user's neck and/or head, or provide relief for apparatuses attached to the user's head, as will be discussed in greater detail herein. In this way, the support device 102 may take numerous shapes and sizes not limited by the embodiments depicted herein. Support device 102, and other support device embodiments described herein, define a first surface and a second surface, wherein the user's head, neck, back, or shoulders are positioned on the first surface, and the second surface of support device 102 is placed proximate a surface such as a bed, stretcher, hospital bed, sofa, or the like.

The support device 102 can be constructed using a variety of materials, including synthetic and natural fabrics, foams and other materials and natural/synthetic blends. The support device 102 may be made from allergy barrier materials comprising, but not limited to, tightly woven barriers, coated barriers to prevent mold spores, pollen, dust mites, and other contaminants from entering through the support device 102. The gauge measurement of the support device 102 (i.e., the number of needles per inch or per 1½ inches in a knitting machine) may be large enough to ensure a finer knit.

The filler material of the support device 102 provides resiliency and support to the support device 102. In other words, the filler material allows the support device 102 to conform to the user's shape and is resilient enough to regain its original shape. The filler material can be fabricated as a single part or multiple parts. In some embodiments the filter material comprises two or more parts. The filler material can be made from one or more materials, including in some embodiments a loose material such as fiber or poly beads. The filler material can comprise polyfiber, reticulated foam, memory foam, hollow petrochemical beads, expanded polystyrene beads or any other natural or synthetic materials that collectively allow the filler material to have the resilient, conforming and supporting properties even after repeated/extended use.

In some embodiments the filler material and the filter barrier (optionally enclosing the filler material) are made of materials that have a high permeability and high wickability. As used herein, wickability means the ability of a fiber or a fabric to disperse moisture and allow it to pass through to the surface of the fabric, so that evaporation can take place. In some embodiments the filler material and the filter barrier comprise performance fabrics to provide functional qualities like moisture management, UV protection, anti-microbial, thermoregulation and wind and water resistance. In some embodiments the filter barrier comprises a mesh. As used herein, a mesh is a type of fabric characterized by its net-like open appearance, and the spaces between the yarns. In some embodiments the filler material and the filter barrier comprise a bacteriostat, fungal stat or antimicrobial material. As used herein, a bacteriostat means a material that kills the bacteria, slows growth or holding the death to growth rates of bacteria more or less in equilibrium, and/or inhibits bacteria growth. As used herein, a fungal stat means a material that kills the fungus, slows growth or holding the death to growth rates of fungus more or less in equilibrium, and/or inhibits fungus growth.

Respiratory apparatuses 100, 200, 600 comprise flexible hose 104. In some embodiments of the invention, flexible hose 104 may be any standard tubing, hose, vacuum or pressure-rated hose, or the like which is configured to be flexible. Flexible hose 104 is substantially hollow and cylindrical in nature and configured to attach to a mask (not pictured) and provide mobility and flexibility to the user wearing a mark of a respiratory device on the user's face. As such, flexible hose 104 may comprise materials such as flexible PVC, polyethylene, nylon, or the like. In some embodiments, flexible hose 104 may have internal helical coils to provide structural stability to flexible hose 104. In other embodiments, flexible hose 104 may be structured to have helical or circular coils made of plastic, rubber, or substantially the same material as flexible hose 104, the purpose of which is to prevent collapse of flexible hose 104 in circumstances where flexible hose 104 is subjected to small a small bend radius or is subjected to an external force capable of collapsing flexible hose 104 otherwise. In other embodiments, however, flexible hose 104 may be smooth and be defined by wall thicknesses and/or materials sufficient to preclude the need for such features. The hollow and cylindrical shape of flexible hose 104 allows for gas to pass through the space defined by the interior walls of flexible hose 104. Furthermore, flexible hose 104 may be a semi-rigid hose, configured to be self-supporting and not be flexible when used in nominal lengths such as between 6 and 24 inches. As such, flexible hose 104 may be configured to be flexible at longer lengths. Flexible hose 104 may be structured to be able to maintain a fixed position, such that flexible hose 104 is articulated by a user to a certain shape, and thereafter flexible hose 104 maintains said shape until further articulating forces are applied to flexible hose 104. In such embodiments, individual sections of flexible hose 104 may rotate independently of adjacent sections of flexible hose 104, whereinafter bellows or hinges integral to each adjacent section of flexible hose 104 lock into various positions independently of one other. Flexible hose 104 may be structured to be removable by a user, such that the user may disconnect flexible hose 104 from respiratory apparatuses 100, 200, 600.

Opposite the user end of flexible hose 104, flexible hose 104 may be mechanically coupled to adapter 108. Adapter 108 may serve one or more of a variety of functions, including (i) converting flexible hose 104 into a rigid tubing system (vertical hose 110 and extension hose 112 which will be described later), and (ii) providing a mechanical capturing point for resilient member 106. Adapter 108 may comprise a clamping or locking mechanism or may simply attach to flexible hose 104 using a barb fitting or quick-connect fitting.

Embodiments of the invention may comprise a resilient member 106, structured to provide adjustability, support, or customization of positioning of flexible hose 104. Resilient member 106 provides additional stability to flexible hose 104, such that flexible hose may be positioned vertically above support device 102. Resilient member 106 may be advantageous in situations where the user may wish to position flexible hose 104 in a location which is comfortable for the user and the user's sleep position during operation of the respiratory apparatus 100. In some embodiments, resilient member 106 may prevent unwanted damage to the connection between flexible hose 104 and adapter 108. Furthermore, some embodiments of resilient member 106 may allow resilient member 106 to return to the natural position of resilient member 106 and as such provide consistent placement of flexible hose 104 relative the support device 102.

In some embodiments such as the embodiment depicted in FIG. 1a-1f, resilient member 106 may be a member with a broken surface area such as a metal wire-like component configured in a spiral or helix shape structured in such a way to enclose and/or capture the outer diameter of flexible hose 104 along at least a portion of the length of flexible hose 104.

In some embodiments resilient member 106 may be configured with a broken surface area in a spiral or helix shape and comprise a soft and/or malleable metal which is capable of being bent and adjusted to various positions, and maintain said positions, without the flexible hose 104 and resilient member 106 returning to the previous positions unless the user adjusts resilient member 106 to those positions. In other embodiments of the invention, resilient member 106 may comprise a spring-like material such as spring steel, such that resilient member 106 is configured to predominantly serve as a damper or shock absorber such that a user applying force to flexible hose 104 would encounter physical resistance provided by the opposite forces from resilient member 106.

In other embodiments of the invention, resilient member 106 may comprise flexible helical conduit. Resilient member 106 may a flexible or semi-flexible material, defined by a substantially circular cross-section, wherein in a spiral or helix shape body is formed integrally with the wall structure of the flexible conduit, such that resilient member 106 is configured in a spiral or helix shape. The spiral or helix shape body may be inserted into the material of resilient member 106 during the manufacturing of resilient member 106.

In some embodiments, resilient member 106 may comprise a flexible and articulating conduit such as a those defined by a plurality of members, each member comprising a male and a female portion of a ball-and-socket joint and an aperture. The resilient member 106 comprises a plurality of these members with the female portion of each member mechanically coupled and rotatably connected to the male portion of the adjacent member, such that resilient member 106 may be articulated and positioned in any number of ways. The apertures of each member align with each adjacent member, such that flexible hose 104 or gas may flow through the series of adjacent apertures.

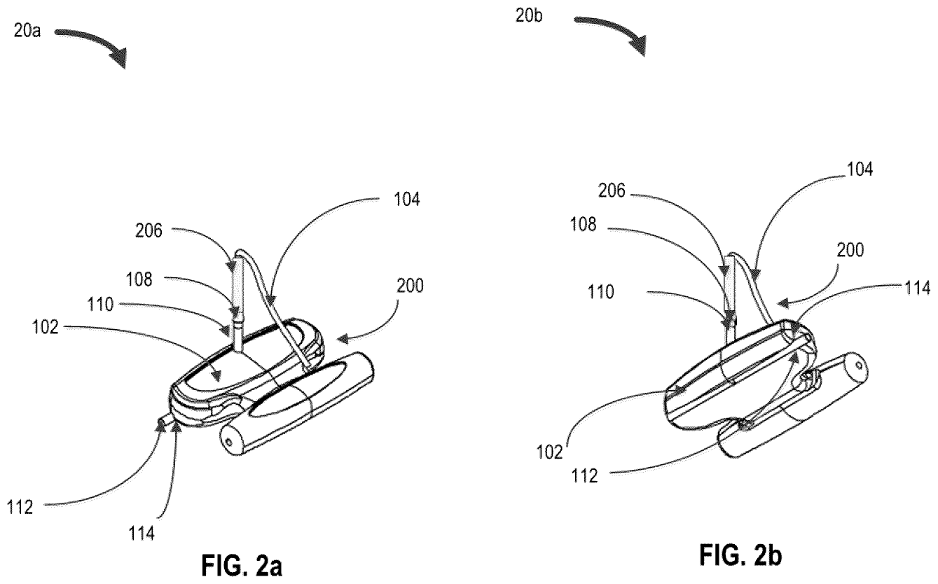

In other embodiments such as those depicted in FIG. 2a-2b, resilient member 206 may not be structured as a helix or spiral, but instead be structured as a length of continuous sleeve defined by a continuous substantially-circular cross-section and continuous surface area, wherein the inner diameter of the resilient member 206 is positioned proximate the outer diameter of flexible hose 104. In some embodiments, resilient member 206 may be structured as a length of continuous sleeve defined by a non-continuous substantially-circular cross-section and broken surface area, whereby the inner and/or outer diameters of the resilient member 206 vary along the length of resilient member 106, such as to create sections and/or lengths of resilient member 206 with varying flexural rigidities by varying the cross-sectional densities of material, wall thickness, diameters, and so forth. In some embodiments, resilient member 206 may be structured as a length of continuous sleeve defined by a continuous substantially-circular cross-section and continuous surface area, whereby the inner and/or outer diameters of the resilient member 206 taper along the length of resilient member 206, such as to create gradient sections and/or lengths of resilient member 206 with gradient flexural rigidities by varying the outer and/or inner diameters of resilient member 206.

Additional embodiments may comprise a resilient member 106 which is formed integrally with flexible hose 104, such that resilient member 106 is configured in a spiral or helix shape, as previously described, however resilient member 106 may be inserted into the material of flexible hose 104 during the manufacturing of flexible hose 104, such that resilient member 106 and flexible hose 104 are inseparable. Additional embodiments of various resilient members are described further in subsequent figures.

In some embodiments, resilient member 206 may be structured to be placed internally to flexible hose 104, such that resilient member 206 has substantially similar characteristics to embodiments where resilient member 206 is positioned externally to flexible hose 104 but is hidden from sight from the user. In this way, the outer diameter of resilient member 206 is positioned proximate the inner diameter of flexible hose 104.

Resilient member 206 may be defined by a higher durometer material than the material of flexible hose 104, or in some embodiments the durometer of resilient member 206 may be variable or gradient along the length of resilient member 206, such that the durometer at the position proximate the support device 102 may be higher that the durometer of the remainder of resilient member 206, or vice-versa. The material of resilient member 206 in such embodiments may be substantially the same material as flexible hose 104, or instead the material of resilient member 106 may be comprised of a different material altogether, including but not limited to polymers, rubbers, metals, mesh composites, wire mesh, ceramics, or the like.

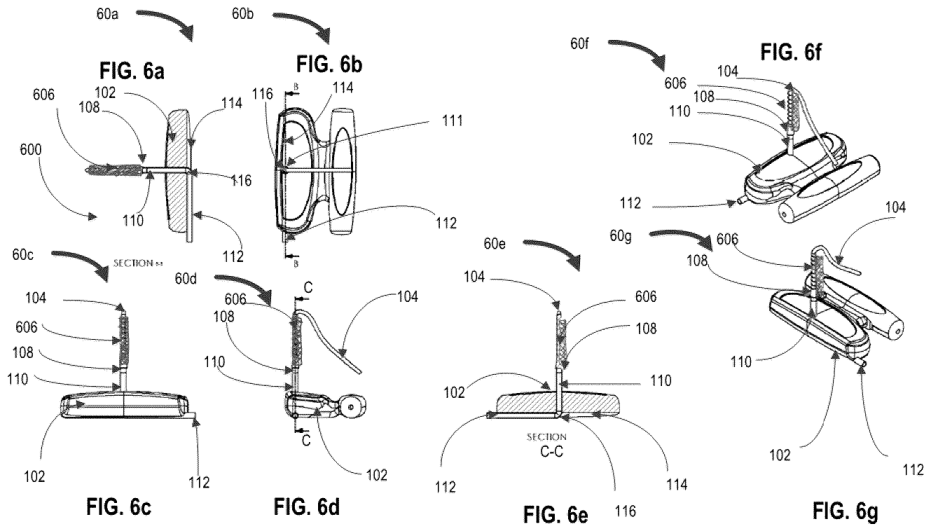

In some embodiments such as those illustrated in FIGS. 6a-6g, resilient member 606 may comprise a mesh-like woven material such as to perform similar functions to resilient members 106, 206. The mesh-like woven material may be formed of a substantially flat or round metal, plastic, rubber, or the like, such that additional support structure is added to flexible hose 104 around the outer diameter of flexible hose 104. In this way, resilient member 606 may be placed in a position, and resilient member 606 retains the position in which is has been placed. In other embodiments, resilient member 606 may be structured to return to its natural position subsequent the removal of any outside forces on resilient member 606.

Mechanically coupled to adapter 108 is vertical hose 110. Vertical hose 110 in the embodiments presented herein may be semi-rigid or rigid tubing such as PVC pipe, acetal, HDPE pipe, or the like. Vertical hose 110 may be preferably in a length between 6 and 24 inches. In other embodiments, vertical hose 110 is the same or similar flexible hose comprising flexible hose 104. Regardless of the material type, vertical hose 110 may be structured to be removable by the user, such that the user may remove vertical hose 110 for cleaning or reconfiguration purposes. Vertical hose 110 is positioned vertically in the respiratory apparatus 100 such that it may be placed within an aperture 111 of the support device 102. Aperture 111 may be substantially circular in shape, and forms a continuous thru-hole feature from the first surface of support device 102 (defined by the interface between the user and the support device 102) and a second surface of support device 102, opposite the first surface. In some embodiments, aperture 111 may be substantially square, triangular, or any such shape capable of accepting vertical hose 110. The second surface of support device 102 is typically positioned such that the second surface rests against a support mechanism such a mattress, secondary pillow, sofa, chair, stretcher, or the like. In some embodiments, aperture 111 is smaller in diameter (or other primary dimension, depending on the shape) than vertical hose 110. In this way, insertion of vertical hose 110 forms a compression fitting with aperture 111. The material of support device 102 immediately adjacent to aperture 111 is compressed by the insertion of vertical hose 110 into aperture 111, and spring-like normal forces from said compression of material are sufficient to support vertical hose 110 in a sturdy manner, such that vertical hose 110 is minimized. In other embodiments, diameter (or other primary dimension, depending on the shape) of aperture 111 may be substantially larger or the same size as vertical hose 110, such that vertical hose 110 is easily removable for cleaning, disassembly, relocation of respiratory apparatus 100, or the like.

In some embodiments, vertical hose 110 may extend beyond the first surface of support device 102 at a height of H1. In other embodiments, vertical hose 110 may not extend past the first surface of support device 102 and may instead be flush or recessed within support device 102. The height H1 may be adjustable such that various lengths of vertical hose 110 may be substituted into the respiratory apparatus 100 depending on the specific preferences of the user, dimensions of support device 102, or the like.

Although vertical hose 110 extends from the first surface of support device 102 to the second surface of support device 102, the second surface of support device 102 may be configured with channel aperture 114 defined by an aperture extending from a first end toward a second end of support device 102. In some embodiments, channel aperture 114 is configured such that at least a portion of channel aperture 114 is exposed along the second surface of support device 102. When a cross section of channel aperture 114 is viewed from either the first end or second end of support device 102, or any plane substantially parallel thereto, channel aperture 114 may be substantially circular in shape, or may be triangular, rectangular, or square or a non-uniform configuration.

In some embodiments, and in particular those embodiments where vertical hose 110 comprises a rigid material, vertical hose 110 is mechanically coupled to angle fitting 116. Angle fitting 116 in some embodiments is a substantially 90-degree fitting which is configured to accept vertical hose 110 and transform the direction of flow of gas which is supplied to and from vertical hose 110. Angle fitting 116 may have a cross section substantially circular when viewed along a plane normal to the flow direction of gas through angle fitting 116. Either end of angle fitting 116 may comprise a female fitting, configured to accept the exterior surface of vertical hose 110 or extension hose 112 within the inner mating surface of angle fitting 116, or it may comprise a male fitting, wherein the exterior surface of the male fitting is configured to accept the interior surface of vertical hose 110 or extension hose 112. In some embodiments, the angle fitting may comprise pressure fittings, barbed fittings, threaded fittings, or the like.

Extending in a direction substantially 90 degrees of rotation from vertical hose 110 is extension hose 112, both of which are mechanically coupled to angle fitting 116 to provide a leak-proof seal between vertical hose 110, extension hose 112, and angle fitting 116. As such, gas which is either under positive pressure or negative pressure (e.g., vacuum), will flow through extension hose 112 and vertical hose 110 (and angle fitting 116 positioned between extension hose 112 and vertical hose 110) without significant leakage of gas. In some embodiments, vertical hose 110 and extension hose 112 may form a singular uniform body, such that the uniform body is continuous and does not require angle fitting 116. For example, a length of rigid or flexible tubing may be mechanically bent or formed at a desired angle prior to or during installation into support device 102 and angle fitting 116 is not required.

As previously described, channel aperture 114 is configured such that channel aperture 114 extends from a first end toward a second end of support device 102. Channel aperture 114 may also be open/exposed along the second surface of support device 102, such that extension hose 112 is accessible from the second surface of support device 102. In one embodiment, the channel aperture 114 extends from the first end to the second end of support device 102. Furthermore, extension hose 112 may be oriented to extend in the direction of the first end of support device 102 or the second end of support device 102. In this way, the user may position extension hose 112 in a configuration which suits the user. For example, the portions of a user's respiratory device which connect to extension hose 112 may be positioned on the left side of the user, so to reduce the amount of hose from the respiratory device to the respiratory apparatus 100, the user may wish to orient the extension hose 112 such that extension hose 112 exits the respiratory apparatus 100 on the left side of the user (e.g., the first end of support device 102). Alternatively, the portions of a user's respiratory device which connect to extension hose 112 may be positioned on the right side of the user, so to reduce the amount of hose from the respiratory device to the respiratory apparatus 100, the user may wish to orient the extension hose 112 such that extension hose 112 exits the respiratory apparatus 100 on the right side of the user (e.g., the second end of support device 102). Extension hose 112 is configured to extend beyond either the first end or the second end of support device 102 and achieve mechanical coupling with a user's respiratory device. The method of coupling to the respiratory device may comprise push-fit connectors, threaded connectors, barbed fittings, luer lock fittings, or the like.

FIGS. 2a-2b illustrate perspective views of a non-limiting example of respiratory apparatus 200, in accordance with another embodiment of the invention. Here, respiratory apparatus 200 comprises resilient member 206 structured as a length of continuous sleeve, as previously described. Resilient member 206 defined by a continuous substantially circular cross-section and continuous surface area, wherein the inner diameter of the resilient member 206 is positioned proximate the outer diameter of flexible hose 104. Resilient member 206 may be structured to provide adjustability, support, or customization of positioning of flexible hose 104. Resilient member 206 may additionally provide stability to flexible hose 104, such that flexible hose may be positioned vertically above support device 102. It shall be understood that all embodiments of resilient member 206 as described herein may be capable of adaptation to respiratory apparatus 200, as well as other respiratory apparatuses described herein.

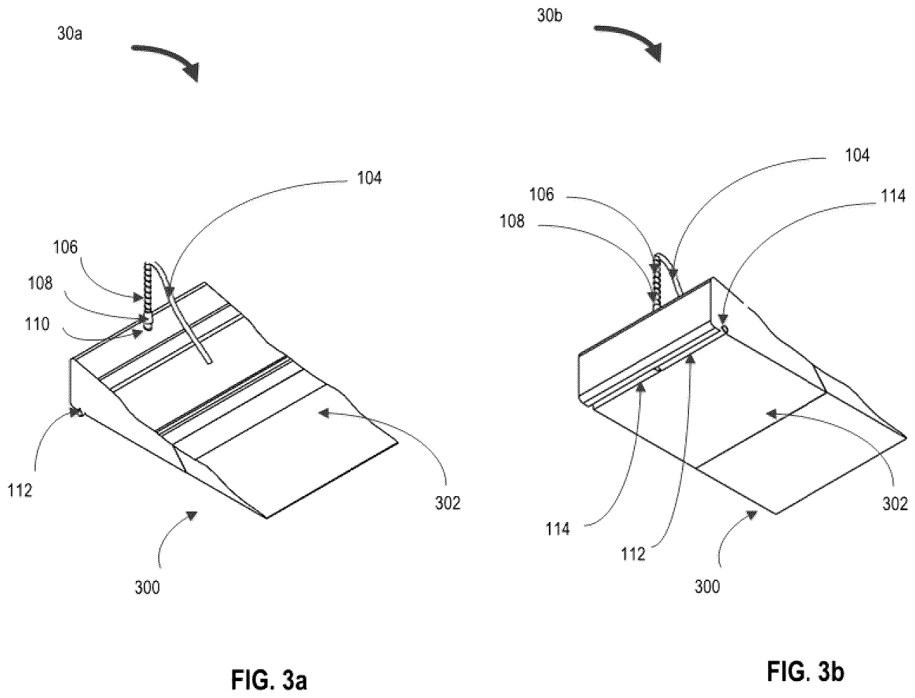

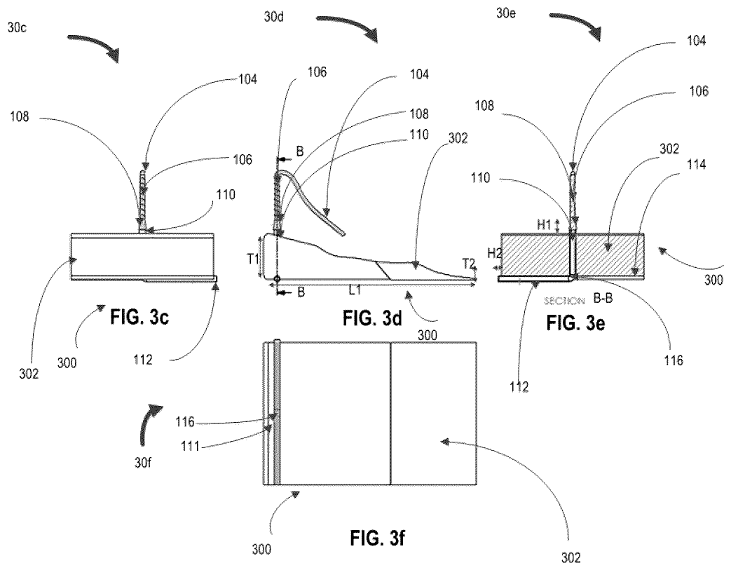

FIGS. 3a-3f illustrate a non-limiting example of respiratory apparatus 300, in accordance with another embodiment of the invention. The respiratory apparatus 300 comprises a support device 302, as illustrated by FIGS. 3a-3f. It shall be noted that like-numbered components throughout this disclosure refer to like-components as described in any of the embodiments. As such, the reader is encouraged to refer to the previous paragraphs to understand the form, structure, and function of any numbered components referenced below which have been previously referenced.

Referring to FIGS. 3a-3f, support device 302 of respiratory apparatus is configured to provide support to one or more of the head, neck, back, or shoulders of a user. Specifically, support device 302 is configured to additionally position the user such that the user's upper body is at an incline relative to the user's lower body. The accomplish this, support device 302 is defined by an upper thickness T1 and a lower thickness T2, such that T1 is greater than T2. In this way, an inclined support device 302 is formed as illustrated in FIG. 2d. The intermediate thicknesses between T2 and T1 along length L1 may range in value to define a straight line or complex curvature between T2 and T1. Support device 302 may comprise additional concave or convex features along the first surface of support device 302 such that an ergonomic shape is formed to better interface with the shoulders, neck, back, and/or head of the user. The material, structure, and features comprising support device 302 are similar to that of which was previously described in another embodiment for support device 102, including channel aperture 114, aperture 111, or the like.

Support device 302 is not limited to the structure as depicted graphically herein, and may take the form of a mattress, mattress topper, or other cushioning and non-cushioning surface sleep elements.

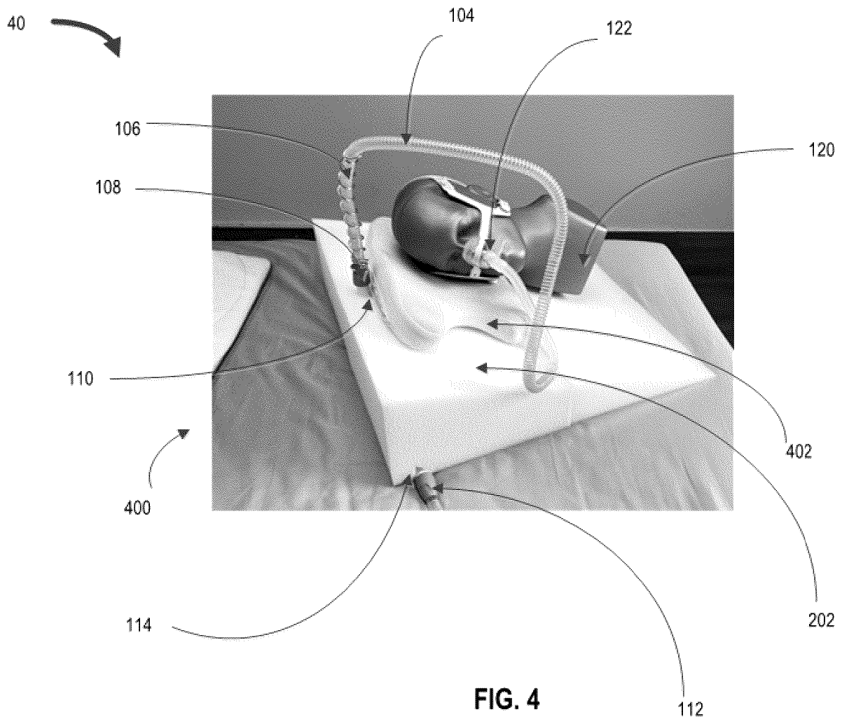

FIG. 4 illustrates a non-limiting example of a respiratory apparatus 400, in accordance with another embodiment of the invention. The respiratory apparatus 400 comprises a support device 202 along with a supplemental pillow 402. Some users 120 prefer to sleep on their side (commonly referred to as side-sleeping). As such, these users 120 often struggle with comfortably wearing a mask connected to a respiratory device during sleep. In this embodiment of the invention, the respiratory apparatus 400 is configured with a supplemental pillow 402, which is placed between the user 120 and the support device 202 (or in some embodiments, support device 102, for any number of different shapes of support device 102). Supplemental pillow 402 may comprise material similar or dissimilar to that of support device 202, and may be mechanically coupled to support device 202 with coupling devices such as Hook-and-loop, buttons, snaps, or the like, or supplemental pillow 402 may be free-floating with respect to support device 202. Furthermore, supplemental pillow 402 may be configured with concave portions, recesses, or the like, capable of providing clearance and relief to the user's mask of the respiratory device and/or hoses attached thereto.

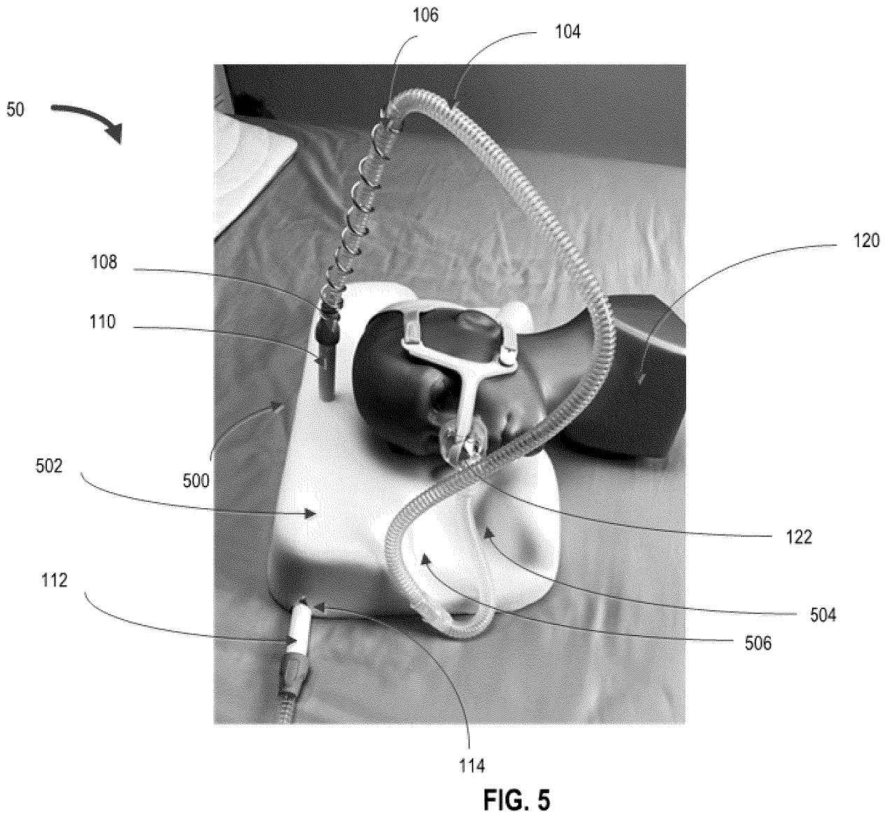

FIG. 5 illustrates a non-limiting example of a respiratory apparatus 500, in accordance with another embodiment of the invention. The respiratory apparatus 500 comprises a support device 502. In this embodiment, support device 502 is structured to accommodate side-sleeping, such that a concave depression is positioned on the first surface of support device 502, extending from the first surface towards the second surface of support device 502. The concave depression is structured to provide relief to the flexible hose 104 and the mask 122 of a user 120. The concave depression may be defined by an inner curve 504 and outer curve 506, such that the distance between the inner curve 504 and outer curve 506 remains substantially constant between the first end and/or second end of the support device 504 and the user 120, when the user 120 is positioned in the center of the first surface of the support device 504.

FIG. 6a-6g illustrate a non-limiting example of respiratory apparatus 600, in accordance with another embodiment of the invention, comprising support device 102 and resilient member 606, both of which have been previously described herein.

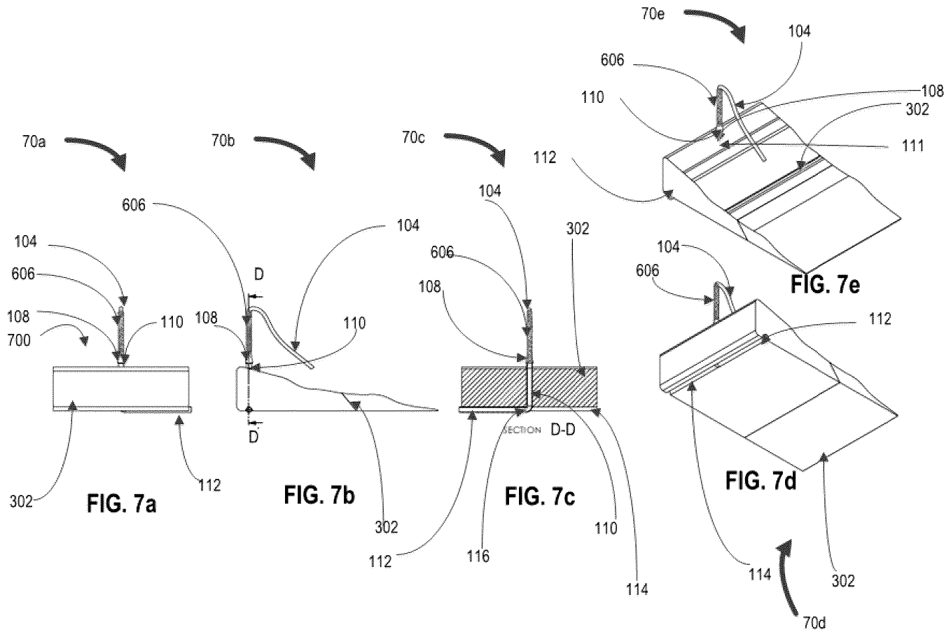

FIG. 7a-7g illustrate a non-limiting example of respiratory apparatus 700, in accordance with another embodiment of the invention, comprising support device 302 and resilient member 606, both of which have been previously described herein.

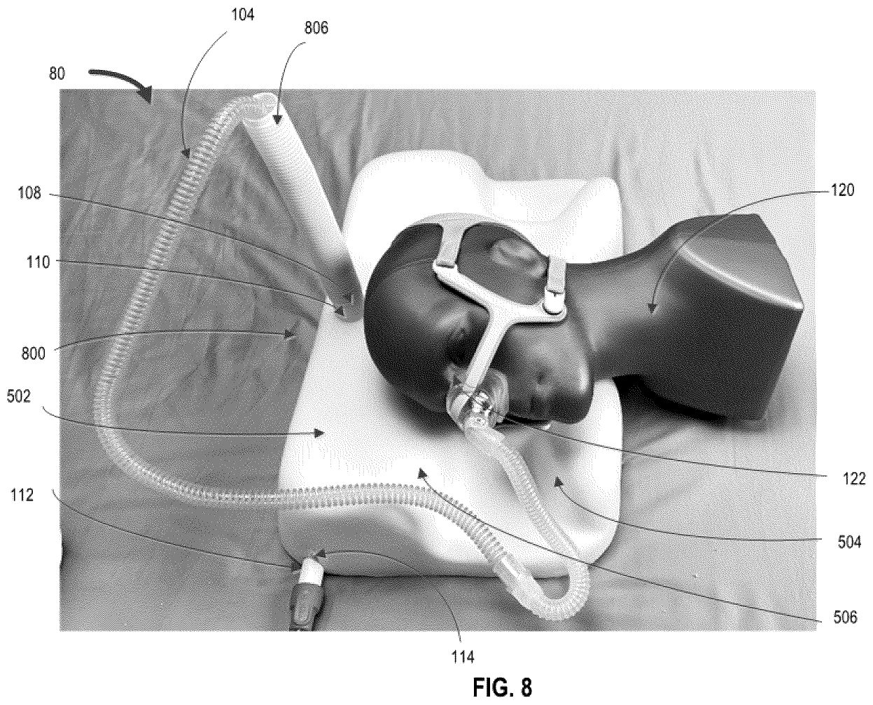

FIG. 8 illustrates a non-limiting example of respiratory apparatus 800, in accordance with another embodiment of the invention, comprising support device 502 and resilient member 806. Resilient member 806 may be structured as a length of continuous sleeve defined by a non-continuous substantially-circular cross-section and broken surface area, whereby the inner and/or outer diameters of the resilient member 806 vary along the length of resilient member 106, such as to create sections and/or lengths of resilient member 806 with varying flexural rigidities by varying the cross-sectional densities of material, wall thickness, diameters, and so forth. As such, resilient member 806 may have the propensity to remain positioned in a way in which user 120 has positioned resilient member 806, or in other embodiments resilient member 806 may be structured to provide structural support sufficient to flex under force from flexible hose 104, but return to the original position after the forces have been removed.

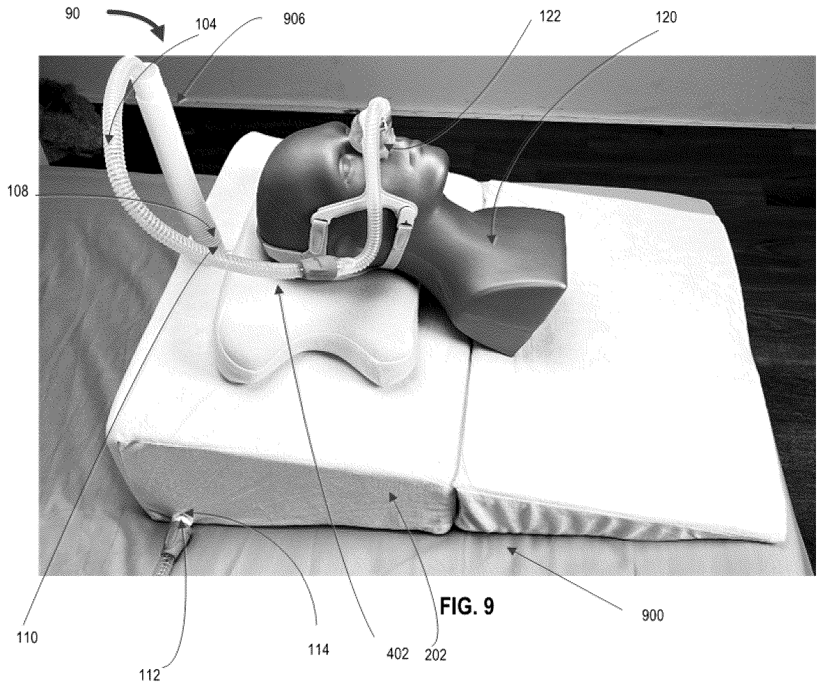

FIG. 9 illustrates a non-limiting example of respiratory apparatus 900, in accordance with another embodiment of the invention, comprising support device 202, supplemental pillow 402, and resilient member 906. In this embodiment, resilient member may be structured as a length of continuous sleeve defined by a continuous substantially-circular cross-section and continuous surface area, wherein the inner diameter of the resilient member 906 is positioned proximate the outer diameter of flexible hose 104. The material of resilient member 906 may be rigid or semi-rigid, such that it supports flexible hose 104 and any forces acted upon resilient member 906 by flexible hose 104.

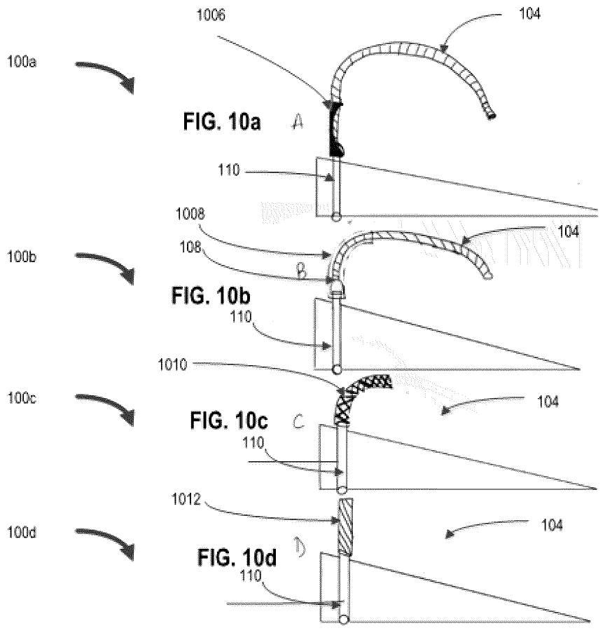

FIG. 10a-10d illustrate several non-limiting examples of resilient members 1006, 1008, 1010, and 1012, in accordance with other embodiments of the invention. As illustrated in FIG. 10a, resilient member 1006 may be structured as a length of continuous sleeve defined by a continuous substantially-circular cross-section, wherein a scalloped portion of resilient member 1006 has been removed such that flexible hose 104 is exposed within resilient member 1006. Resilient member 1006 may comprise any number of aforementioned materials or construction methods, such that resilient member 1006 is either rigid or semi-rigid, and either retains the shape in which it has been placed, or returns to its natural shape after a force to resilient member 106 has been applied and removed.

As illustrated in FIG. 10b, resilient member 1008 may be structured as a length of continuous sleeve defined by a continuous substantially-circular cross-section, wherein one end of resilient member 1008 is structured to overlap adapter 108, such that the outer diameter of adapter 108 is enclosed within the inner diameter of resilient member 1008.

As illustrated in FIG. 10c, resilient member 1010 may be structured as a length of continuous sleeve defined by a continuous substantially-circular cross-section, wherein one end of resilient member 1010 may be structured to overlap adapter 108, such that the outer diameter of adapter 108 may be enclosed within the inner diameter of resilient member 1010. Resilient member 1010 may comprise a mesh-like woven. The mesh-like woven material may be formed of a substantially flat or round metal, plastic, rubber, or the like, such that additional support structure is added to flexible hose 104 around the outer diameter of flexible hose 104. In this way, resilient member 1010 may be placed in a position, and resilient member 1010 retains the position in which is has been placed. In other embodiments, resilient member 1010 may be structured to return to its natural position subsequent the removal of any outside forces on resilient member 1010.

As illustrated in FIG. 10d, resilient member 1012 may be structured as a length of continuous sleeve defined by a continuous substantially-circular cross-section, wherein one end of resilient member 1012 may be structured to overlap adapter 108, such that the outer diameter of adapter 108 is enclosed within the inner diameter of resilient member 1012. In this embodiment, resilient member 1012 may be structured as a length of continuous sleeve defined by a continuous substantially-circular cross-section and continuous surface area, wherein the inner diameter of the resilient member 1012 is positioned proximate the outer diameter of flexible hose 104. The material of resilient member 1012 may be rigid or semi-rigid, such that it supports flexible hose 104 and any forces acted upon resilient member 1012 by flexible hose 104.

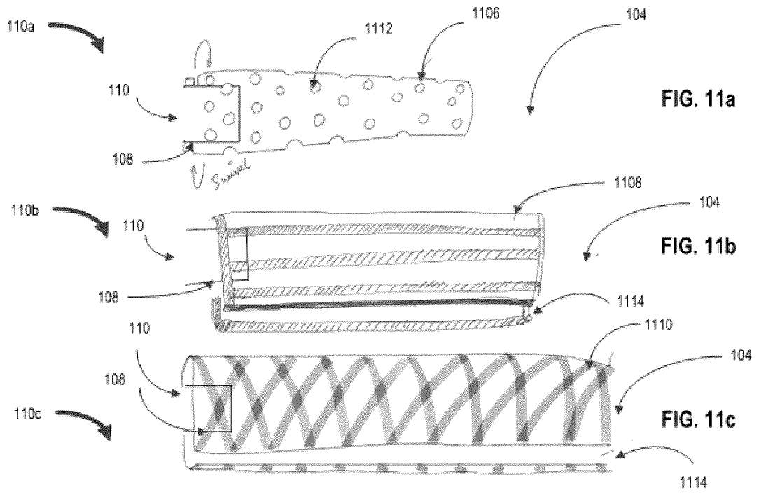

FIG. 11a-11c illustrate several non-limiting examples of resilient members 1106, 1108, and 1110, in accordance with other embodiments of the invention. As illustrated in FIG. 11a, resilient member 1106 may be structured as a length of continuous sleeve defined by a continuous substantially-circular cross-section, wherein one end of resilient member 1106 is structured to swivel around adapter 106. Resilient member 1106 may comprise a plurality of apertures 1112 extending from the outer surface of resilient member 1106 to the inner surface of resilient member 1106, such that resilient member 1106 is lightweight, flexible, or the like. Resilient member 1106 may comprise plastic, foam, rubber, metal, or a combination thereof.

As illustrated in FIG. 11b, resilient member 1108 may comprise a plurality of substantially parallel wires, each wire with a circular, rectangular, or other polygonal cross section, comprising materials such as metal, plastic, rubber, or the like. At a first end of resilient member 1108, each of the first end of the plurality of wires is connected to a first substantially-circular support wire, such that resilient member 1108 retains an overall cylindrical structure on the first end. At a second end of resilient member 1108, each of the second end of the plurality of wires is connected to a second substantially-circular support wire, such that resilient member 1108 retains an overall cylindrical structure on the second end. A slit 1114 may be applied to resilient member 1108, such that the slit 1114 extends from the first end of resilient member 1108 to the second end of resilient member 1108, allowing for the placement of resilient member 1108 around flexible hose 104 without removal of an device attached thereto.

As illustrated in FIG. 11c, resilient member 1110 may be structured as a length of continuous sleeve defined by a continuous substantially-circular cross-section, wherein one end of resilient member 1110 may be structured to overlap adapter 108, such that the outer diameter of adapter 108 is enclosed within the inner diameter of resilient member 1110. In this embodiment, resilient member 1110 may be structured as a length of continuous sleeve defined by a continuous substantially-circular cross-section and continuous surface area, wherein the inner diameter of the resilient member 1110 is positioned proximate the outer diameter of flexible hose 104. The material of resilient member 1110 may be rigid or flexible closed-cell or open-cell foam. A slit 1114 may be applied to resilient member 1110, such that the slit 1114 extends from the first end of resilient member 1110 to the second end of resilient member 1110, allowing for the placement of resilient member 1110 around flexible hose 104 without removal of an device attached thereto.

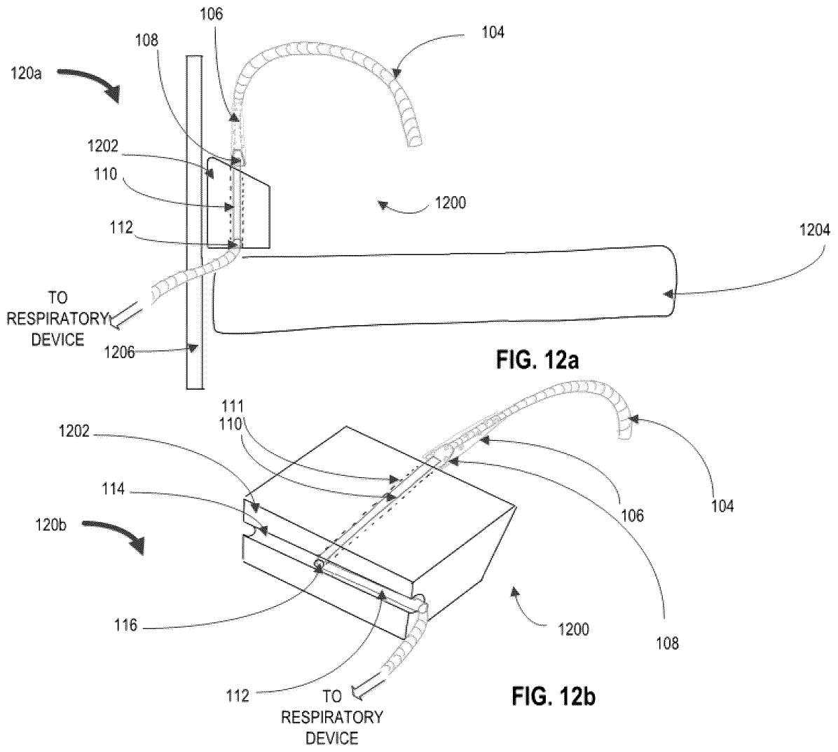

FIGS. 12a-12b illustrate a non-limiting example of respiratory apparatus 1200, in accordance with another embodiment of the invention. It shall be noted that, as is the case with all other previous embodiments, resilient member 106 may be used in some embodiments of respiratory apparatus 1200, but may not be used in other embodiments of respiratory apparatus 1200. In this embodiment, support device 1202 is configured to be adjacent a vertical surface 1202. Vertical surface 1202 may be a headboard, wall, temporary vertical structure, IV pole, or the like. Attachment of support device 1202 to vertical surface 1202 may be through any number of mechanical fasteners, including but not limited to hook-and-loop material, snaps, hooks, glue, fasteners, or the like. Support device 1202 may also not be attached to vertical surface 1202, but instead may simply rest on horizontal surface 1204. Horizontal surface 1204 may be a bed, sofa, hospital bed, stretcher, chair, recliner, or any number of surfaces on which a user may located. Respiratory apparatus 1200 is primarily structured for supporting and/or routing various respiratory device accompaniments including but not limited to flexible hose 104, vertical hose 110, adapter 108, extension hose 112, or the like. As such, respiratory apparatus 1200 may not be structured to primarily support portions of the body of the user. As depicted in FIGS. 12a-12b, support device 1202 is defined by channel aperture 114 and aperture 111.

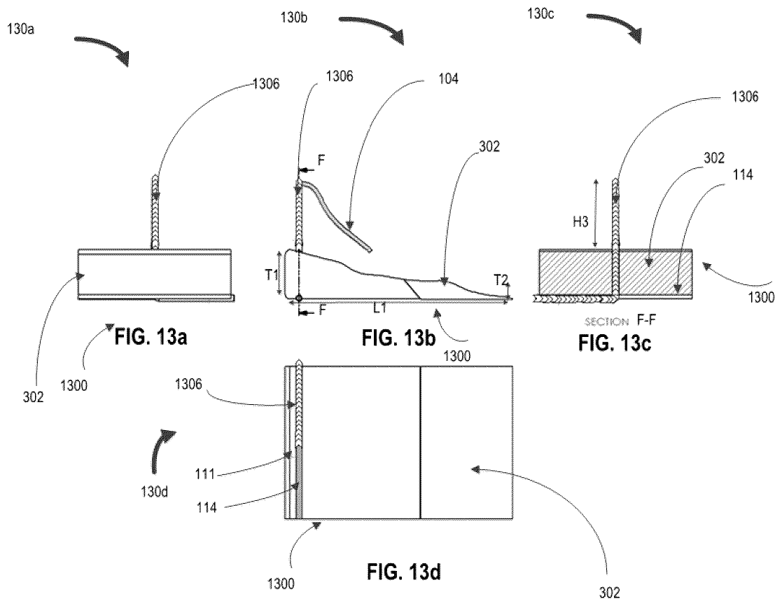

FIGS. 13a-13d illustrate a non-limiting example of respiratory apparatus 1300, in accordance with another embodiment of the invention. As illustrated in FIGS. 13a-13d, it shall be noted that in some embodiments of the invention, including but not limited to all of the previously described embodiments, flexible hose 104 may not be coupled to an adapter 108, but instead, flexible hose 104 may be mechanically coupled to resilient member 1306. Resilient member 1306 may itself be a gas conduit. As such, resilient member 1306 may be coupled to extension hose 112, or entirely replace one or more of extension hose 112, vertical hose 110, adapter 108, and angle fitting 116. As such, and as shown in FIGS. 13a-13d with respiratory apparatus 1300, resilient member 106 is coupled to the respiratory device and in gaseous communication with the respiratory device. In yet additional embodiments not depicted graphically, resilient member 1306 may be coupled to adapter 108 or directly to vertical hose 110.

Although not shown graphically in FIGS. 13a-13d, resilient member 1306 may comprise one or more coupling attachments on either end of resilient member 1306, configured to direct the flow of gas between resilient member 1306 and the respiratory device or flexible hose 104 without significant leakage of gas. These coupling attachments may be push-connect fittings, barbed fittings, swaged fittings, rotatable/swiveling fittings, quick connect fittings, or the like. In this way, a minimal amount of flexible hose 104 is necessary for the apparatus, and additional gas conduits may be unnecessary. As is the case in the embodiment of FIGS. 13a-13d, the usage of one or more of vertical hose 110, adapter 108, and extension hose 112 may not be needed, and as such are not included in the apparatus.

Resilient member 1306 may comprise a flexible and articulating conduit such as a those defined by a plurality of members, each member comprising a male and a female portion of a ball-and-socket joint and an aperture. The resilient member 1306 comprises a plurality of these members with the female portion of each member mechanically coupled and rotatably connected to the male portion of the adjacent member, such that resilient member 1306 may be articulated and positioned in any number of ways. The apertures of each member are in gaseous communication with each adjacent member, such that gas may flow through the series of adjacent apertures.

In other embodiments of the invention, resilient member 1306 may comprise flexible helical conduit. Resilient member 1306 may a flexible or semi-flexible material, defined by a substantially circular cross-section, wherein in a spiral or helix shape body is formed integrally with the wall structure of the flexible conduit, such that resilient member 1306 is configured in a spiral or helix shape. The spiral or helix shape body may be inserted into the material of resilient member 1306 during the manufacturing of resilient member 1306.

In any of the aforementioned embodiments, and as previously discussed with other embodiments of resilient member 1306, resilient member 1306 may be structured to extend in a vertical direction, such that a height H3 may be achieved by resilient member 1306 vertically above support device 102, 302. 402, 502, or the like. In this way, resilient member 1306 is configured to provide adjustability, support, or customization of positioning of flexible hose 104 to conform with a user's position.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. In addition, where possible, any terms expressed in the singular form herein are meant to also include the plural form and/or vice versa. As used herein, “at least one” shall mean “one or more” and these phrases are intended to be interchangeable. Accordingly, the terms “a” and/or “an” shall mean “at least one” or “one or more,” even though the phrase “one or more” or “at least one” is also used herein.

What is claimed:

1. An apparatus for routing hose, comprising:
a support device, wherein the support device defines a first surface and an opposite second surface, a first end and an opposite second end, the support device comprising:
an aperture proximate the first surface extending from the first surface to the second surface; and
a channel aperture extending from the first end toward the second end, wherein the channel aperture at least partially defines the second surface; and
a resilient member.

2. The apparatus according to claim 1, wherein the apparatus comprises a hose having an outer surface and wherein the resilient member defines an inner surface, and wherein the inner surface of the resilient member is coupled to an outer surface of the hose.

3. The apparatus according to claim 2, wherein the hose has a first section and a second section, wherein the first and the second sections have different flexibility.

4. The apparatus according to claim 3, wherein the first section comprises a flexible hose and the second section comprises a vertical hose.

5. The apparatus according to claim 4, wherein the vertical hose is positioned within the aperture.

6. The apparatus according to claim 4, wherein a flexible hose is positioned within the aperture.

7. The apparatus according to claim 4, further comprising an extension hose and an angle fitting, and wherein the flexible hose is coupled to the vertical hose, and the angle fitting is coupled to the vertical hose and the extension hose.

8. The apparatus according to claim 1, further comprising an extension hose, wherein the extension hose is positioned proximate the channel aperture.

9. The apparatus according to claim 1, wherein the resilient member is positioned within the aperture and is positioned proximate the channel aperture.

10. The apparatus according to claim 7, wherein the resilient member is releasably coupled to the flexible hose.

11. The apparatus according to claim 1, wherein the resilient member comprises a plurality of a ball-and-socket joints.
